(19) United States
(12) Patent Application Publication
(10) Pub. No.: US 2018/0355346 A1
(43) Pub. Date: Dec. 13, 2018

(54) COMPOSITIONS AND METHODS FOR NUCLEIC ACID PURIFICATION FROM BLOOD SAMPLES

(71) Applicant: Corning Incorporated, Corning, NY (US)

(72) Inventors: Yi-Cheng Hsieh, Horseheads, NY (US); Cheng-l Hsu, Zhubei-si (TW); Chia-Ling Wu, Tainan City (TW); Hsan Jan Yen, New Taipei City (TW)

(21) Appl. No.: 15/757,830

(22) PCT Filed: Sep. 9, 2016
(86) PCT No.: PCT/US2016/050901

§ 371 Date: Mar. 6, 2018

Related U.S. Application Data
(60) Provisional application No. 62/217,144, filed on Sep. 11, 2015.

(51) Int. Cl. C12N 15/10; C12Q 1/6806

(52) U.S. Cl. CPC C12N 15/1013; C12Q 1/6806

(57) ABSTRACT

Provided herein are systems, kits, and methods for nucleic acid purification from blood samples. In particular, provided herein are reagents, e.g. dissolving buffer comprising sarcosine, and methods of using such reagents, for high yield purification of nucleic acids from blood samples, including dried blood samples. Also claimed is a system for purifying nucleic acids from whole blood samples comprising a lysis buffer comprising guanidine and disodium hydrogen phosphate. Magnetic particles are suggested as solid support for capturing the nucleic acids.

This application claims the benefit of priority under 35 U.S.C. § 119 of U.S. Provisional Application Ser. No. 62/217,144 filed on Sep. 11, 2015, the content of which is relied upon and incorporated herein by reference in its entirety.

### FIELD

Provided herein are compositions, systems, kits, buffers and methods for nucleic acid purification from blood samples. In particular, provided herein are reagents, and methods of using such reagents, for high yield purification of nucleic acids from blood samples, including dried blood samples.

### BACKGROUND

Newborn screening is an assay for identifying problems which affect the survival or health of babies after birth. Millions of newborn babies routinely receive the test in the U.S every year (cdc.gov website, newborn screening page). Heel blood of newborn babies is collected from 24 hours to 7 days after birth by specialized filter papers, a technique that was introduced by Robert Guthrie in 1963 and started to be used in 1970s, and shipped to a laboratory for metabolite and enzyme activities analysis. Metabolites and enzyme activities are examined from whole blood samples for most newborn screening. Metabolism dysfunctions, genetic disorders or hearing loss can be detected through this screening, such as phenylketonuria, sickle cell disease, and lysosomal storage disorders (Gelb M H et al., 2006; Guthrie R and SusiA, 1963; Giordano P C, 2009; Michlitsch J et ai., 2009; Streetly A et al., 2008, herein incorporated by reference in its entirety). Recently, the CDC has worked to expand genomic-related tests for newborn screening program such as cystic fibrosis, diabetes, and birth defects. More and more newborn screening labs are adding DNA-based testing, including confirmatory testing of positive results of diseases and expansion of disorder testing.

Using such approaches, nucleic acids have to be extracted from dried blood samples and then amplified prior to analysis. Thus, the quality and quantity of the extracted genomic DNA (gDNA) have to meet the requirements for PCR or other amplification techniques. Various protocols, such as methanol-, CHELEX-100- and Tris-EDTA-based methods were developed for extracting gDNA for downstream applications. Table 1 lists a few examples of existing products for purifying gDNA from dried blood and whole blood samples.

Among these products, are those that employ spin columns and magnetic bead-based purification, some of which are amenable to automation. Magnetic bead-based approaches can reduce the need for centrifugation.

TABLE 1
Examples of genomic DNA purification kits for blood samples.

| Product Format | Product Name | Company |
|---|---|---|
| Spin column | Dried Blood Spot (DBS) | Norgen |
| | DNA Isolation Kit | |
| | QIAamp DNA Mini Kit | Qiagen |
| | QIAamp ™ DNA | |
| | Investigator Kit | |
| | PureLink ™ Genomic DNA | Invitrogen |
| | Mini Kit | |
| Magnetic particles | DNA IQ ™ System | Promega |
| | MagaZorb ® DNA Mini- | |
| | Prep Kit | |
| | EZ1 DNA Tissue Kit | Qiagen |

To have higher quality of nucleic acid purification, Boom et al. developed a buffer system based on guanidine thiocyanate together with silica in 1990 (Boom R, Sol C J, Salimans M M, Jansen C L, Wertheim-van Dillen P M, van der Noordaa J. Rapid and simple method for purification of nucleic acids. J Clin Microbiol. 1990 March; 28(3):495-503; herein incorporated by reference in its entirety) (8.3 M guanidine thiocyanate, 82 mM Tris, 36 mM EDTA, 2% Triton X-100). Silica can bind to nucleic acid from serum, urine, or bacteria samples in the presence of high guanidine thiocyanate concentration and release nucleic acid under low salt conditions. Based on Boom's protocol, Schneeberger's group also developed a new and specified buffer system for DNA purification from dried blood spot (DBS) samples (lysis buffer I: 10 mM Tris-HCl (pH 8.0), 2 mM EDTA, 50 mM NaCal, 2% SDS; lysis buffer II: 120 g GuSCN in 100 ml of 0.1 M Tris-HCl (pH 6.4) with 22 ml of 0.2M EDTA (pH 8.0) and 2.6 g of TRITON X-100). More DNA was purified and the quality was sufficient for downstream PCR application as showed in Schneeberger's publication (Schneeberger C, Kury F, Larsen J, Speiser P, Zeillinger R. A simple method for extraction of DNA from Guthrie cards. PCR Methods Appl. 1992; 2:177-9).

Even with these approaches and improvements, there is still a need for further improved compositions and methods for purification of DNA from blood samples. High quality DNA is needed in high amounts that are amenable to the downstream analysis steps used in modern analytical techniques, particularly as the number of tests to be run from a single sample increases.

### SUMMARY

Provided herein are compositions, systems, buffers, kits, and methods for nucleic acid purification from blood samples. In particular, provided herein are buffers, and methods of using such buffers, for high yield purification of nucleic acids from blood samples, including dried blood samples.

For example, in some embodiments, provided herein are systems comprising one or more buffers or other components for purification of nucleic acid from a sample. The system may comprise sets of reagents, for example, in the form of a kit. Kits may include one or more storage or shipping vessels (e.g., tubes, vials, etc.) housing a buffer and one or more containers (e.g., boxes) housing the storage or shipping vessels, along with other components (e.g., instruction for use, magnets, stirring components, etc.) that may be used in a method described herein.

In some embodiments, the systems comprise one or more or each of: a) a dissolving buffer; b) a lysis buffer; and c) a nucleic acid capture solid support (e.g., surface, resin, column, bead (e.g., magnetic beads such as paramagnetic beads)). In some embodiments, the dissolving buffer comprises a surfactant. In some embodiments, the dissolving buffer comprises a surfactant, a chelating agent, and a base. In some embodiments, the lysis buffer comprises a protein denaturant and/or a salt. In some embodiments, the solid support binds nucleic acid molecules under defined conditions (e.g., charge, solubility, etc.). In some embodiments, the solid support comprises an affinity capture molecule. In some embodiments, the solid support does not comprise an affinity capture molecule.

In some embodiments, the surfactant comprises, consists of, or consists essentially of an ionic surfactant. In some embodiments, the ionic surfactant comprises, consists of, or consists essentially of an ionic surfactant derived from sarcosine, such as N-lauroylsarcosine. In some embodiments, the N-lauroylsarcosine is provided as a salt, such as sodium [dodecanoyl(methyl)amino]acetate. Any useful concentration of the surfactant may be used, which may be altered as desired to accommodate particular types of samples. In some embodiments, the surfactant is present in the first buffer at from 0.05-10% by volume (e.g., 0.1%, 0.5%, 1%, 2%, 3%, 4%, 5%, 6%, 7%, 8%, 9%, 10% or any values or ranges therein between, e.g., 2.1, 2.2, 2.3; e.g., 2-3, 3-4, 2.5-4, etc.). In some embodiments, the concentration of the surfactant is about 3% or is 3%. As used herein, unless specified otherwise, “about” refers to +/−10% of a recited value.

In some embodiments, the base comprises, consists of, or consists essentially of a primary amine. In some embodiments, the base comprises, consists of, or consists essentially of Tris. Any useful concentration of the base may be used, which may be altered as desired to accommodate particular types of samples. In some embodiments, the base is present in the first buffer at a concentration from 1-20 mM (e.g., 1 mM, 2 mM, 3 mM, 4 mM, 5 mM, 6 mM, 7 mM, 8 mM, 9 mM, m10 M, 11 mM, 12 mM, 13 mM, 14 mM, 15 mM, 16 mM, 17 mM, 18 mM, 19 mM, 20 mM or any values or ranges therein between, e.g., 8.1, 8.2, 8.3; e.g., 8-11, 9-12, 8.5-20, etc.). In some embodiments, the concentration is about 10 mM or is 10 mM.

In some embodiments, the chelating agent comprises, consists of, or consists essentially of EDTA. In some embodiments, the chelating agent is provided as a salt, e.g., EDTA 2NA or EDTA 4NA. Any useful concentration of the chelating agent may be used, which may be altered as desired to accommodate particular types of samples. In some embodiments, the chelating agent is present in the first buffer at a concentration from 0.05 to 2.0 mM (e.g., 0.05 mM, 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, or any values or ranges therein between, e.g., 0.81, 0.82, 0.83; e.g., 0.05-1.1, 0.9-2.0, 0.1-1.0, etc.). In some embodiments, the concentration is about 1.0 mM or is 1.0 mM.

In some embodiments, the dissolving buffer further comprises one or more components that denature or destroy undesired contaminants, such as proteins. In some embodiments, such a component is a protease. In some embodiments, the protease is proteinase K. In some embodiments the dissolving buffer is a combined dissolving/lysis buffer.

In some embodiments, the protein denaturant of the lysis buffer comprises, consists of, or consists essentially of a chaotropic agent. In some embodiments, the chaotropic agent comprises, consists of, or consists essentially of guanidine thiocyanate (GITC), guanidine hydrochloride (GuHCL), and combinations thereof. Any useful concentration of the protein denaturant may be used, which may be altered as desired to accommodate particular types of samples. In some embodiments, the protein denaturant is present in the lysis buffer at a concentration from 2-6 M (e.g., 2 M, 3 M, 4 M, 5 M, 6 M, or any values or ranges therein between, e.g., 4.1, 4.2, 4.3; e.g., 2-5, 4-6, 4.5-5, etc.). In some embodiments, the concentration is about 5.0 M or is 5.0 M.

In some embodiments, the salt of the lysis buffer comprises, consists of, or consists essentially of a salt useful in adjusting the pH of the buffer to a desired value or range. In some embodiments, the salt of the lysis buffer comprises, consists of, or consists essentially of a phosphate salt. In some embodiments, the phosphate salt is a sodium phosphate. In some embodiments, the sodium phosphate is a disodium phosphate. In some embodiments, the disodium phosphate is a disodium phosphate hydrate. Any useful concentration of the salt may be used, which may be altered as desired to accommodate particular types of samples. In some embodiments, the salt is present in the second buffer at a concentration from 90-110 mM (e.g., 90 mM, 100 mM, 104 mM, 110 mM, or any values or ranges therein between, e.g., 103, 104, 105; e.g., 90-105, 100-110, 102.5-110, etc.). In some embodiments, the concentration is about 104 mM or is 104 mM.

In some embodiments, the system comprises, consists of, or consists essentially of: a lysis buffer, said lysis buffer comprising guanidine thiocyanate and/or disodium hydrogen phosphate; and a solid support (e.g., paramagnetic beads). In some embodiments, the system further comprises a lysis buffer comprising a surfactant, a chelating agent, and a base.

In some embodiments, the system comprises, consists of, or consists essentially of a dissolving buffer comprising N-lauroylsarcosine sodium salt; and a lysis buffer comprising guanidine thiocyanate. In some embodiments, the dissolving buffer further comprises a base and a chelating agent. In some embodiments, the dissolving buffer further comprises one or more components that denature or destroy proteins. In some embodiments, the lysis buffer further comprises a salt. In some embodiments, the system further comprises a nucleic acid capture solid support (e.g., bead (e.g., magnetic beads such as paramagnetic beads)).

In some embodiments, the above systems further comprise one or more wash buffers. In some embodiments, one or more of the wash buffers comprises an alcohol. In some embodiments, the alcohol is ethanol. In some embodiments, the alcohol is isopropanol (IPA). In some embodiments, one or more of the wash buffers comprises an alcohol (e.g., ethanol, isopropanol, etc.) and lysis buffer. In some embodiments, one or more of the wash buffers comprises an alcohol (e.g., ethanol, isopropanol, etc.), lysis buffer, and dissolving buffer. In some embodiments, one or more of the wash buffer consists of water and ethanol. In some embodiments, the ethanol is present at 60-80% by volume (e.g., 60%, 70%, 80% or any values or ranges therein between, e.g., 71, 72, 73; e.g., 60-75, 70-80, 65.5-75.5, etc.). In some embodiments, the concentration is about 70% or is 70%. In some embodiments, the wash buffer comprises GuHCl, EDTA, Tris, and isopropanol (e.g., W1A buffer: 3.98 M GuHCl, 26 mM EDTA.4Na, 20 mM Tris, 10% IPA).

In some embodiments, the systems further comprise a device or component for magnetically isolating magnetic beads. In some embodiments, such device or component comprises a magnet. Any of a wide variety of stands, plates, instruments or other devices that are commercially available may be employed.

In some embodiments, the systems further comprise a test sample (i.e., a sample containing or suspected of containing nucleic acid to be purified). In some embodiments, the test sample is a blood sample. In some embodiments, the blood sample is a dried blood sample. In some embodiments, the test sample comprises blood in filter paper (e.g., blood spots dried on filter paper). In some embodiments, the systems comprise one or more control samples (e.g., positive or negative control samples). Positive control samples include, but are not limited to, samples known to include nucleic acid, blood samples, purified nucleic acid in a suitable storage buffer, and the like. Where the purified nucleic acid samples are to be analyzed for forensic, diagnostic, research, or other indications, the positive control may include a specific nucleic acid useful as a positive control in the subsequent analysis (e.g., a gene sequence comprising a mutation; an infectious disease nucleic acid; a disease biomarker; etc.). In some embodiments, one or more positive control samples are provided having a known concentration of nucleic acid to assist in the quantitation of nucleic acid amount in a test sample or to assess limits of detection of a particular assay. Negative control samples include, but are not limited to, samples lacking nucleic acid.

In some embodiments, the system comprises a reaction mixture which is, for example, a test sample such as whole blood or a control sample (e.g., a positive or negative control sample) combined with one or more of the buffers and/or other components (e.g., beads).

Further provided herein are methods of purifying nucleic from a sample. In some embodiments, the methods comprise use of any of the buffers or other system components described above, alone or in any desired combination, to purify nucleic acid from a sample. In some embodiments, the sample is contacted (e.g., via mixing) with the lysis buffer. In some embodiments, the sample is first contacted with the dissolving buffer to generate a dissolved sample. The dissolved sample is then contacted with the lysis buffer. In some embodiments, the sample is a blood sample (e.g., a dried blood sample or a whole blood sample). In some embodiments, the method further comprises the step of treating the sample with a solid support (e.g., paramagnetic beads) to generate support-bound nucleic acid. In some embodiments, the method further comprises the step of washing the support-bound nucleic acid with one or more wash solutions. In some embodiments, multiple washes are employed using one or more wash solutions. In some embodiments a first and/or subsequent wash employs a solution comprising at least a portion of the dissolving and/or lysis buffer and ethanol. In some embodiments, the wash buffer comprises a chaotropic agent and alcohol (e.g., W1A buffer). In some embodiments, a later wash employs an alcohol solution. In some embodiments, the alcohol solution comprises ethanol (e.g., 60-80% ethanol). Where magnetic beads are employed, the beads may be restrained by a magnet during the wash steps to maintain the beads containing the nucleic acid in the reaction vessel. In some embodiments, the method further comprises the step of contacting the support-bound nucleic acid with an elution buffer (e.g., TE buffer) to generate eluted nucleic acid. In some embodiments, the method achieves a high yield of purified nucleic acid. For example, in some embodiments, the method obtains at least 40 ng (e.g., at least 50 ng, at least 60 ng, at least 70 ng, at least 80 ng, at least 90 ng, or any value or range between those values; e.g., from 40-90 ng, etc.) of gDNA from a starting sample of three 3 mm diameter dried blood spots.

Purified target nucleic acid (e.g., DNA (for example, genomic DNA)) may be analyzed using one or more analytical techniques or may be used for therapeutic, diagnostic, research, or other desired indications. In some embodiments, subsequent use of the purified nucleic acid involves technique including, but not limited to, amplification (e.g., via PCR, TAQMAN, or other amplification techniques), sequencing (e.g., via next-generation sequencing methods or other sequencing techniques), hybridization analysis (e.g., via in situ methods such as FISH, microarray analysis, or other probe-based hybridization techniques), mass spectroscopic analysis, or any other desired technique.

It should be understood that where the terms “comprising,” “comprises,” “having,” and other open-ended terms are used herein, embodiments are also contemplated where the system, device, or method may alternatively consist of or consist essentially of the recited elements.

Where methods are described, unless specified otherwise or by necessity, it should be understood that the recited method steps can be conducted in any order or simultaneously.

### DESCRIPTION OF FIGURES

FIG. 1 is a flowchart showing an embodiment of a method for gDNA purification from DBS.

FIG. 2A-G is a schematic representation of the method shown in FIG. 1.

FIG. 3 is a flowchart of gDNA purification from whole blood using an embodiment of the technology described herein.

FIG. 4 A-F is a schematic representation of the method shown in FIG. 3.

FIG. 5 shows a graph comparing DNA yield (in nanograms) of DNA obtained from dried blood spot (DBS) samples using an embodiment of the technology described herein (CRCT) compared to technologies from commercially available vendors.

FIG. 6 shows a graph comparing DNA yield (in nanograms) of DNA obtained from whole blood samples using an embodiment of the technology described herein (CRCT) compared to technologies from commercially available vendors.

FIG. 7 shows data obtained from nucleic acid purification using embodiments of the technology described in Example 4 comparing different surfactants.

FIG. 8 shows data obtained from nucleic acid purification using embodiments of the technology described in Example 4 comparing different surfactants and alcohol concentrations.

FIGS. 9 A and B show data obtained from nucleic acid purification using embodiments of the technology described in Example 4 comparing different surfactants and alcohols.

FIG. 10 shows data obtained from nucleic acid purification using embodiments of the technology described in Example 4 comparing different relative buffer concentrations.

FIG. 11 shows data obtained from nucleic acid purification using embodiments of the technology described in Example 4 comparing different GuSCN and ethanol concentrations.

FIG. 12 shows data obtained from nucleic acid purification using embodiments of the technology described in Example 4 comparing different buffer compositions.

FIG. 13 shows data obtained from nucleic acid purification using embodiments of the technology described in Example 4 comparing different binding buffer pH values.

FIG. 14 shows data obtained from nucleic acid purification using embodiments of the technology described in Example 4 comparing different buffer compositions and processing steps.

### DETAILED DESCRIPTION

Provided herein are compositions, systems, buffers, kits, and methods for nucleic acid purification from blood samples. In particular, provided herein are buffers, and methods of using such buffers, for high yield purification of nucleic acids from blood samples, including dried blood samples.

For example, provided herein are compositions and methods employing buffer systems, bead-based isolation of nucleic acid, and wash and elution buffers that out-perform industry-leading products currently on the market. Side-by-side experiments conducted herein demonstrate a yield of purified gDNA from dried blood samples more than twice that of the QIAamp DNA mini kit (Qiagen) and approximately 5-times that of the DNA IQ system (Promega). The obtained nucleic is high quality and amendable to use in down-stream analytical methods and the process is automation-friendly and cost-effective in that it can be conducted without centrifugation or vacuum steps.

In some embodiments, provided herein is a buffer system for obtaining high quality, high yield DNA from sample such as blood samples, including from dried blood samples. In some embodiments, where dried blood samples are employed, the buffer system comprises a dissolving buffer to facilitate removal of sample from filter paper containing the dried blood spot. In such embodiments, the buffer system may further comprise a lysis buffer to facilitate release of nucleic acid from other components of the sample. In some embodiments, where the sample is whole blood, the dissolving buffer may be skipped and the sample may be processed directly with the lysis buffer.

In some embodiments, the desired nucleic acids are released from a sample (e.g., dried blood in filter paper) and undesired components (e.g., proteins) are destroyed (e.g., digested) by one or more contaminant-removing agents in the dissolving or lysis buffers (e.g., a protease such as proteinase K, a denaturant such as guanidine thiocyanate, etc.).

In some embodiments, the compositions and methods comprise the use of solid supports (e.g., beads, resins, columns, surfaces, etc.) for purification of nucleic acid. In some embodiments, the solid support comprises, consists essentially of, or consists of paramagnetic beads. In some embodiments, the beads are paramagnetic silica beads. In some embodiments, a binding buffer enhances the binding capacity of paramagnetic silica beads for nucleic acid (e.g., from dried blood spots or whole blood samples). In some embodiments, the binding buffer is composed of a mixture of the dissolving buffer and/or lysis buffer with an alcohol such as ethanol.

In some embodiments, one or more wash steps are employed with one or more wash buffers to remove contaminants (e.g., proteins, cellular debris, etc.) and/or to remove salts. In some embodiments one or more first washes employ a wash solution comprising dissolving buffer and/or lysis buffer with an alcohol (e.g., ethanol). In some embodiments, the wash buffer comprises a chaotropic agent and alcohol (e.g., W1A buffer). In some embodiments, one or more subsequent wash steps employ a wash buffer consisting of an alcohol (e.g., ethanol) and water.

In some embodiments, an elution buffer is employed to elute purified nucleic acid that is attached to the beads. The purified nucleic may then be used for any desired purpose.

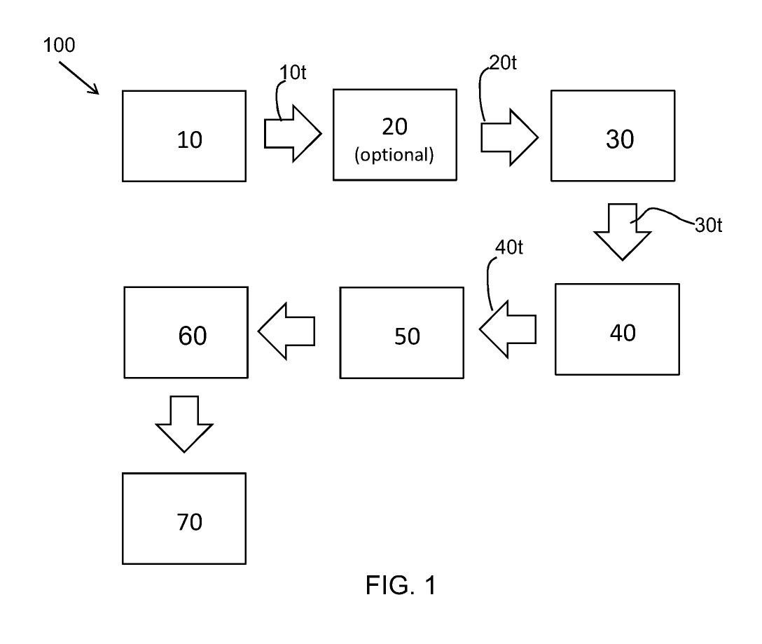

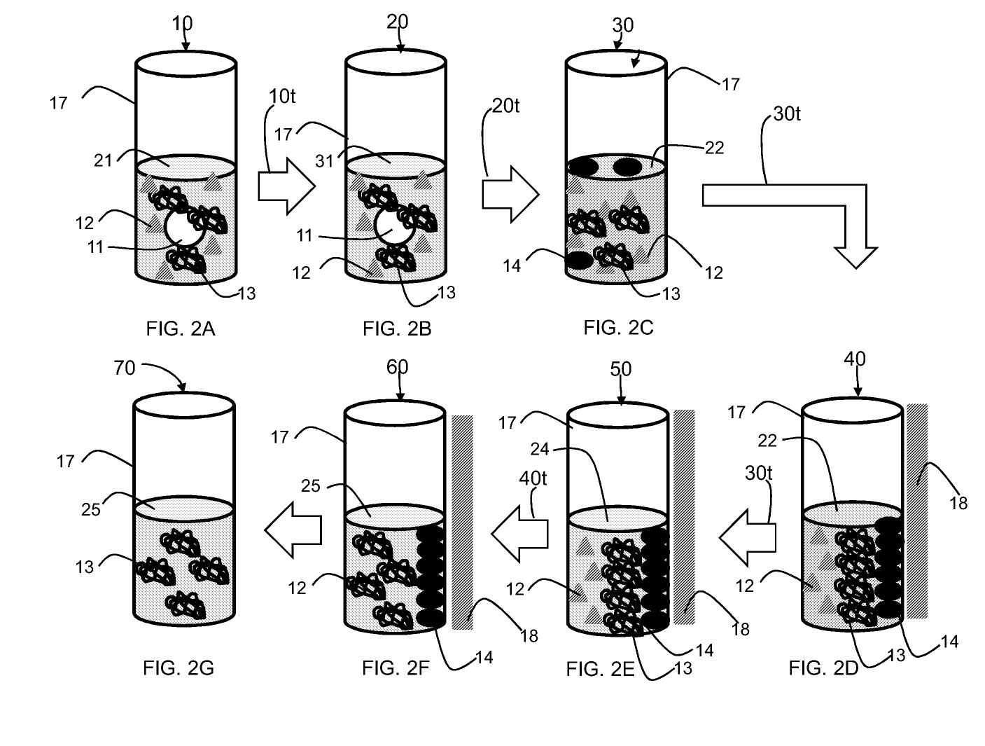

FIG. 1 is a flowchart showing an exemplary protocol 100 for use in purifying nucleic acid from dried blood spots 11 (DBS, see FIG. 2 in an embodiment. FIG. 2A-G is a schematic representation of the method shown in FIG. 1. FIG. 1 and FIG. 2 will be described together.

In a first step 10, one or more (e.g., 1, 2, 3, 4, 5, etc.) samples of dried blood 11, which, in embodiments, may be dried on pieces of filter paper as shown in FIG. 2, embodiments may be on a piece of filter paper 19, is added to a reaction vessel 17 along with a dissolving buffer 21. In the embodiment shown in FIG. 1, the dissolving buffer 21 separates the dried blood from the filter paper. In step 10, the sample, the DBS 11 is incubated for a time period 10t (e.g., 30 min) in the dissolving buffer 21. Alternatively, a buffer (e.g., dissolving buffer) may be flushed through the filter paper to remove desired sample.

In embodiments, an optional lysis buffer 31 may be employed, as shown in step 20 of FIGS. 1, and 20 in FIG. 2B. In embodiments, the lysis buffer 31 may be the dissolving buffer 21, with a lysis component added to it. Or, in embodiments, the dissolving buffer 21 may be removed between step 10 and step 20, and a lysis buffer 31 added to the container 17. This may be accomplished by spinning down the contents of the container 17, removing the supernatant, and resuspending the resulting pellet in a separate lysis buffer 31. The lysis component digests undesired material 12 or contaminants 12 such as proteins or other cellular components. When a lysis buffer 20 (FIG. 2B) is employed, the lysis buffer is then incubated for a time period 20t (e.g. 30 min) in the lysis buffer 31.

Next, a binding step 30 is conducted as shown in FIG. 2C. In embodiments, the binding buffer is formed from the addition of alcohol to the dissolving buffer 21 of step 10, or to the optional lysis buffer 31 of step 20. Nucleic acid capture solid supports 14 which may be magnetic or paramagnetic beads are also added to the binding buffer 22. As shown in Step 30 of FIG. 1 and FIG. 2C, the binding buffer 22 contains nucleic acid capture solid supports 14 which, in embodiments may be magnetic beads or paramagnetic beads in addition to the cellular components; the targets 13, the undesired components 12 such as cellular debris such as proteins, the binding buffer 22 and. The sample is incubated for a time period 30t to allow the target 13, the nucleic acid of interest, to associate with the beads 14.

Next, in step 40, as shown in FIG. 2D, the reaction vessel 17 containing the binding buffer 22, targets 13, undesired material 12 and magnetic or paramagnetic beads 14, is placed in proximity to a magnetic field (e.g., from a magnet 18) to separate beads 14 away from the remainder of the sample containing undesired materials 12 (e.g., cell debris, protein, etc.). The sample is incubated for a time 40t (e.g., 5 min).

Next, in step 50 and as shown in FIG. 2E, wash buffer is added to the vessel 17. The liquid contained in the vessel may be removed. Undesired material 12, will be suspended in the wash buffer 24 while target material, associated with nucleic acide capture solid supports or magnetic beads 14, will remain on the side of the vessel 17, because of the presence of a magnet 18. This wash step may be repeated several times to remove undesired material 12.

Next, in step 60 as shown in FIG. 2F, an elution buffer is added to disassociate the purified target or nucleic acid from the nucleic acid capture solid support or beads. This elution step may be incubated for a time period 60t (e.g., 5 minutes) to complete the elution of the target material 13.

Next, in step 70 and as shown in FIG. 2G, the solution is spun down and the supernatant containing the target DNA is removed. Nucleic acid capture solid supports or beads 14 will form a pellet and can be reused or discarded. As shown in FIG. 2G, the result of the protocol is isolated target DNA 13. The resulting solution provides a purified nucleic acid product.

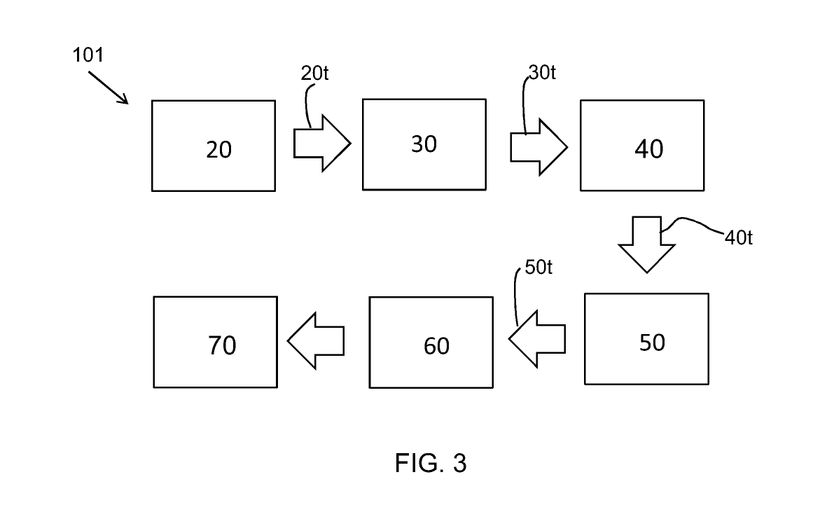

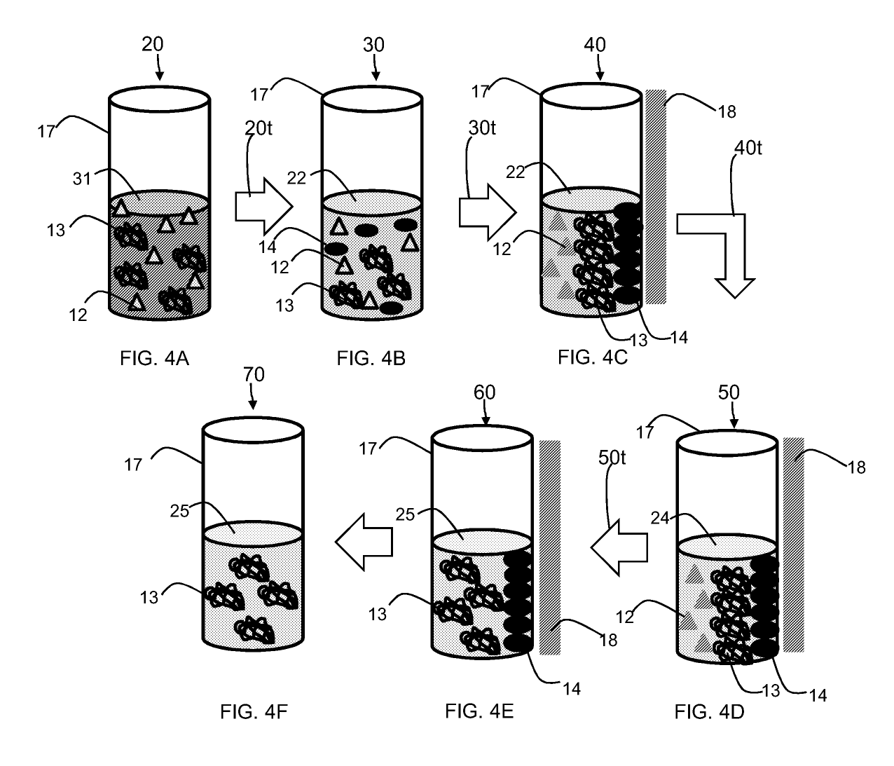

FIG. 3 is a flow chart illustrating an exemplary protocol for use in purifying nucleic acid from whole blood and FIG. 4A-F are schematic representations of the method shown in FIG. 3. FIG. 3 and FIG. 4 will be described together. When purifying nucleic acid from whole blood, no filter paper or other substrate having dried blood is necessary, and therefore no step to dissolve dried blood from the substrate is necessary. Therefore, the first step, step 10 shown in FIG. 1 and FIG. 2A is not necessary. Instead, a lysis step to break up whole blood cells is the starting point for this embodiment.

As shown in FIG. 3 and FIG. 4, in a first step 20 and FIG. 4A, whole blood is added to a reaction vessel 17 along with a lysis buffer. The sample is incubated for a first time period 20t (e.g., 5 min) during which time undesired contaminants 12 such as proteins are digested, and target nucleic acid 13 is released into the buffer.

Next, as shown at step 30 in FIG. 3 and FIG. 4B, a binding step is conducted. In an embodiment, alcohol is added to the lysis buffer to form binding buffer 22. Nucleic acide capture solid supports, in this embodiment in the form of magnetic beads 14 are also added. The sample is incubated for a second time period 30t (e.g., 5 min) to allow the nucleic acid of interest 13 to associate with the beads 14.

Next, as shown at step 40 in FIG. 3 and FIG. 4C, the reaction vessel 17 is placed in proximity to a magnetic field (e.g., from a magnet 18) to separate beads away from the remainder of the sample containing undesired materials 12 (e.g., cell debris, protein, etc.). The sample is incubated for a third time period 40t (e.g., 5 min),

Next, as shown at step 50 in FIG. 3 and FIG. 4D, the sample is washed with a wash buffer 24, removing the undesired materials that are not associated with beads 14. The wash step may be repeated.

Next, as shown at step 60 in FIG. 3 and FIG. 4E, an elution buffer 25 is added to disassociate the purified target nucleic acid 13 from the beads 14. The elution buffer solution is incubated for a time period 50t (e.g., 5 min),

Next, as shown at step 70 in FIG. 3 and FIG. 4F, the elution buffer containing the dissociated purified target nucleic acid 13 is removed to separate container 17. The resulting solution provides a pure target nucleic acid product 13.

### Exemplary Buffers and Buffer Components:

In some embodiments, an issolving buffer 21 is provided. The dissolving buffer 21 finds use, for example, in treating dried blood samples (e.g., dried blood present in a material). In some embodiments, the dissolving buffer 21 is prepared by adding component ingredients to water (e.g., distilled water, ddH₂O).

In some embodiments, the dissolving buffer 21 comprises a base. In some embodiments, the base contains a primary amine. In some embodiments the base is Tris (2-amino-2-hydroxymethyl-propane-1,3-diol). In embodiments, the base (e.g., Tris) is present in the buffer at a concentration of from 1-20 mM (e.g., 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, m10 mM, 10.5 mM, 11 mM, 11.5 mM, 12 mM, 12.5 mM, 13 mM, 13.5 mM, 14 mM, 14.5 mM, 15 mM, 15.5 mM, 16 mM, 16.5 mM, 17 mM, 17.5 mM, 18 mM, 18.5 mM, 19 mM, 19.5 mM, 20 mM, or any concentration or range between such values).

In some embodiments, the dissolving buffer 21 comprises a chelating agent. In some embodiments, the chelating agent is 2-({2-[Bis(carboxymethyl)amino]ethyl} (carboxymethyl)amino)acetic acid (Ethylenediaminetetraacetic acid; EDTA). In some embodiments, the EDTA is present in the buffer at a concentration of 0.05 to 2.0 mM (e.g., 0.05 mM, 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM or any concentration or range between such values). In some embodiments, the EDTA is provided as a salt. For example, in some embodiments, the EDTA is provided as EDTA disodium salt (C₁₀H₁₄N₂Na₂O₈.2H₂O) or as EDTA 4Na (C₁₀H₁₂N₂Na₄O₈, 4H₂O). Where both Tris and EDTA are provided, the buffer may be referred to as TE buffer.

In some embodiments, the dissolving buffer 21 comprises a surfactant. In some embodiments, the surfactant is an ionic surfactant. In some embodiments, the surfactant is an ionic (e.g., anionic) surfactant derived from sarcosine. In some embodiments, the surfactant is N-lauroylsarcosine (aka sarcosyl or sarkosyl), for example, provided as a sodium salt (Sodium [dodecanoyl(methyl)amino]acetate). In some embodiments, the N-Lauroylsarcosine sodium salt is present in the buffer at from 1-10% by volume (e.g., 1%, 1.5%, 2%, 2.5%, 3%, 3.5%, 4%, 5%, 6%, 7%, 8%, 9%, 10%, or any concentration or range between such values). In some embodiments, the N-Lauroylsarcosine sodium salt is provided at pH 8.0. In some embodiments, the surfactant is sodium dodecyl sulfate (SDS). In some embodiments, the SDS is present in the buffer at from 0.05-10% by volume (e.g., 0.05%, 0.1%, 0.3%, 0.5%, 1%, 2%, 3%, 4%, 5%, 6%, 7%, 8%, 9%, 10%, or any concentration or range between such values). In some embodiments, the surfactant is a nonionic surfactant. In some embodiments, the surfactant is TRITON X-100 (t-octylphenoxypolyethoxyethanol). In some embodiments, the TRITON X-100 is present in the buffer at from 0.05-10% by volume (e.g., 0.05%, 0.1%, 0.5%, 0.6%, 1%, 2%, 3%, 4%, 5%, 6%, 7%, 8%, 9%, 10%, or any concentration or range between such values). In some embodiments, the surfactant is TX-114 (TRITONX-114; tert-octylphenoxypoly (ethoxyethanol)). In some embodiments, the TX-114 is present in the buffer at from 0.05-10% by volume (e.g., 0.05%, 0.1%, 0.5%, 0.6%, 1%, 2%, 3%, 4%, 5%, 6%, 7%, 8%, 9%, 10%, or any concentration or range between such values).

In some embodiments, the dissolving buffer 21 comprises one or more lysis components that denature or destroy undesired contaminants, such as proteins. In some embodiments, the component is a protease. In some embodiments, the protease is Proteinase K. In some embodiments, the Proteinase K is present in the buffer at a concentration of 50-5000 μg/ml (e.g., 50 μg/ml, 60 μg/ml, 70 μg/ml, 80 μg/ml, 90 μg/ml, 100 μg/ml, 200 μg/ml, 300 μg/ml, 400 μg/ml, 500 μg/ml, 600 μg/ml, 700 μg/ml, 800 μg/ml, 900 μg/ml, 1000 μg/ml, 2000 μg/ml, 3000 μg/ml, 4000 μg/ml, 5000 μg/ml, or any concentration between such values). In some embodiments, the component is a denaturant or reducing agent. In some embodiments, the reducing agent is 2-mercaptoethanol. In some embodiments, the 2-mercaptoethanol is present in the buffer at from 100-1000 mM (e.g., 100 mM, 200 mM, 300 mM, 400 mM, 500 mM, 600 mM, 700 mM, 800 mM, 900 mM, 1000 mM, or any concentration or range between such values).

In embodiments, the dissolving buffer 21 may comprise a base, a chelating agent, a surfactant and optionally one or more lysis components, individually or in combination.

In some embodiments, a lysis buffer 31 is provided. As shown in FIG. 1 and FIG. 2A-G, depending upon the nature of the sample, dissolving buffer 21 may be used to remove a sample from a sample substrate such as filter paper 19. In some embodiments, lysis components may be added to the dissolving buffer 21 to create a combined dissolving/lysis buffer, or dissolving buffer 21 may be replaced with lysis buffer 31. Or, in embodiments as shown in FIG. 3 and FIG. 4A-F, where the sample is not contained in a sample substrate, no dissolving buffer is necessary, and the process may begin with a lysis buffer 31.

In some embodiments, the lysis buffer 31 is prepared by adding lysis components to water (e.g., distilled water, ddH₂O). In some embodiments, the lysis buffer 31 comprises a protein denaturant. In some embodiments, the protein denaturant is a chaotropic agent. In some embodiments, the chaotropic agent is guanidine thiocyanate (C₂H₆N₄S) (aka guanidinium thiocyanate or GITC) or guanidine hydrochloride or combinations thereof. In embodiments, the chaotropic agent is present in the buffer at a concentration of from 2-6 M (e.g., 2M, 2.5 M, 3 M, 3.5M, 4M, 4.5 M, 5 M, 5.5 M, 6M, or any concentration or range between such values).

In some embodiments, the lysis buffer 31 comprises a salt. In some embodiments, the salt is a phosphate. In some embodiments, the salt is a sodium phosphate. In some embodiments, the sodium phosphate is disodium phosphate (Na₂HPO₄). In some embodiments, the disodium phosphate is provided as a hydrate (e.g., Na₂HPO₄.12H₂O). In embodiments, the disodium phosphate is present in the buffer at a concentration of from 90-110 mM (e.g., 90 mM, 95, mM, 100 mM, 104 mM, 105 mM, 110 mM, or any concentration or range between such values). In some embodiments, the disodium phosphate is provided at pH 8.7.

In embodiments, the lysis buffer 31 may comprise a protein denaturant (which may be a chaotropic agent), a salt or a combination.

In some embodiments, the dissolving buffer 21 is added to a sample, followed by addition of the lysis buffer 31 without intervening removal of the first buffer. As such, in some embodiments, a buffer is provided that is a combination or mixture of the dissolving buffer 21 and the lysis buffer 31.

In some embodiments, to facilitate binding of released nucleic acid to beads, a binding buffer 22 is created by adding alcohol to the lysis buffer 31 or the combination of the dissolving 21 and lysis buffers 31. In some embodiments, the alcohol is ethanol (e.g., anhydrous ethanol). In some embodiments, an amount of alcohol is added such that the concentration of alcohol in the binding buffer 22 is from 20%-70% (e.g., 20%, 25%, 30%, 35%, 50%, 70%, or any concentration or range between such values).

In some embodiments, nucleic acid capture solid supports 14 are provided. In embodiments the nucleic acid capture solid supports are magnetic beads 14. Magnetic beads 14 are provided to capture target nucleic acid 13 in the sample. In some embodiments, the beads 14 are magnetic beads. In some embodiments, the beads are paramagnetic beads. In some embodiments, the beads are DYNABEADS® available from ThermoFisher, TURBOBEADS® available from TurboBeads LLC, Zurich Switzerland, asynchronous magnetic beads, or combinations thereof. In some embodiments, the beads are paramagnetic Q beads (MagQu Co. Ltd.). Paramegantic Q beads from MagQu Co. Ltd. are approximately 3 μm in diameter, have a stable magnetic iron oxide core, a highly water-soluble and bio-compatible dextran-coated surface, and allow covalent coupling to surface probes.

In some embodiments, a wash buffer 24 is provided. The wash buffer finds use in removing contaminants from nucleic acid bound to the beads. In some embodiments, the wash buffer comprises an alcohol. In some embodiments, the alcohol is ethanol. In some embodiments, the alcohol is isopropanol. In some embodiments, that wash buffer comprises alcohol and lysis buffer and/or dissolving buffer. In some such embodiments, the wash buffer may comprise the same ingredients as the binding buffer. In some embodiments, the wash buffer comprises, consists, or consists essentially of alcohol and water. In some embodiments, the alcohol (e.g., ethanol) is present at 60-80% by volume (e.g., 60%, 65%, 70%, 75%, 80%, or any concentration or range between such values).

In some embodiments, an elution buffer 25 is provided. The elution buffer 25 finds use to remove target nucleic acid 13 from the beads 14 for collection and use in downstream applications. In some embodiments, the elution buffer 25 comprises a base. In some embodiments, the base contains a primary amine. In some embodiments the base is Tris (2-amino-2-hydroxymethyl-propane-1,3-diol). In embodiments, the base (e.g., Tris) is present in the buffer at a concentration of from 1-20 mM (e.g., 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 8 mM, 8.5 mM, 9 mM, 9.5 mM, 10 mM, 10.5 mM, 11 mM, 11.5 mM, 12 mM, 12.5 mM, 13 mM, 13.5 mM, 14 mM, 14.5 mM, 15 mM, 15.5 mM, 16 mM, 16.5 mM, 17 mM, 17.5 mM, 18 mM, 18.5 mM, 19 mM, 19.5 mM, 20 mM, or any concentration or range between such values). In some embodiments, the elution buffer comprises a chelating agent. In some embodiments, the chelating agent is 2-({2-[Bis(carboxymethyl)amino]ethyl} (carboxymethyl)amino)acetic acid (Ethylenediaminetetraacetic acid; EDTA). In some embodiments, the EDTA is present in the buffer at a concentration of 0.05 to 2.0 mM (e.g., 0.05 mM, 0.06 mM, 0.07, mM, 0.08 mM, 0.09 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, or any concentration or range between such values). In some embodiments, the elution buffer is provided at a pH from 7.0 to 8.0 (e.g., pH 8.0).

In embodiments, one or more of the buffers may be provided in kit form in a container (e.g., tube, vial, etc.) as a concentrate (e.g., 2×, 5×, 10×, 50×, 100× concentrate). For example, where it is desired that a buffer component have a final concentration of 0.1 mM in solution, a 10× concentration may be provided as 1 mM. Addition of 1 μL of the concentrate to a 9 μL of liquid produces the intended 0.1 mM concentration. In some embodiments, multiple buffer components are provided together in a single solution in appropriate relative concentrations.

In an aspect (1), the disclosure provides a system comprising a) a dissolving buffer comprising a surfactant; b) a lysis buffer comprising a protein denaturant; and c) a nucleic acid capture solid support. In another aspect (2), the disclosure provides a system according to aspect (1), wherein said dissolving buffer further comprises a chelating agent and a base. In another aspect (3), the disclosure provides a system according to aspect (1) wherein said lysis buffer further comprises a salt. In another aspect (4), the disclosure provides a system according to aspect (1), wherein said nucleic acid capture solid support comprises a paramagnetic bead. In another aspect (5), the disclosure provides a system according to aspect (1), wherein said surfactant comprises an ionic surfactant. In another aspect (6), the disclosure provides a system according to aspect (5), wherein said ionic surfactant comprises an ionic surfactant derived from sarcosine. In another aspect (7), the disclosure provides a system according to aspect 6, wherein said surfactant comprises N-lauroylsarcosine. In another aspect (8), the disclosure provides a system according to aspect (7), wherein said surfactant comprises sodium [dodecanoyl(methyl)amino]acetate. In another aspect (9), the disclosure provides a system according to any of aspects 1-8, wherein said surfactant is present in the dissolving buffer at from 0.05-10% by volume. In another aspect (10), the disclosure provides a system according to aspect (9), wherein said surfactant is present in the dissolving buffer at 3% by volume. In another aspect (11), the disclosure provides a system according to aspect (1), wherein said dissolving buffer further comprises one or more components that denature or destroy proteins. In another aspect (12), the disclosure provides a system according to aspect (11), wherein said one or more components that denature or destroy proteins comprises proteinase K.

In a further aspect (13), the disclosure provides a system comprising: a) a lysis buffer, said lysis buffer comprising guanidine thiocyanate, guanidine hydrochloride, or combinations thereof and disodium hydrogen phosphate; and b) a nucleic acid capture solid support. In another aspect (14), the disclosure provides a system according to aspect (13), further comprising a lysis buffer comprising a surfactant, a chelating agent, and a base.

In a further aspect (15) the disclosure provides a system comprising: a) a dissolving buffer comprising N-lauroylsarcosine sodium salt; and b) a lysis buffer comprising guanidine thiocyanate. In another aspect (16), the disclosure provides a system according to aspect (15), wherein said dissolving buffer further comprises a base and a chelating agent. In another aspect (17), the disclosure provides a system according to aspect (15), wherein said dissolving buffer further comprises one or more components that denature or destroy proteins. In another aspect (18), the disclosure provides a system according to aspect (15), wherein said lysis buffer further comprises a salt. In another aspect (19), the disclosure provides a system according to aspect (15), further comprising a nucleic acid capture solid support. In another aspect (20), the disclosure provides a system according to aspect (19), wherein said a nucleic acid capture solid support comprises a magnetic bead or a paramagnetic bead.

In a further aspect (21), the disclosure provides the system of any of aspects 1-20, further comprises one or more wash buffers. In a further aspect (22) the disclosure provides the system of any of aspects 1-21, further comprising a magnet. In a further aspect (23), the disclosure provides the system of any one of aspects 1-22, further comprising a test sample. In a further aspect (24), the disclosure provides the system of aspect (23), further comprising a blood sample. In a further aspect (25), the disclosure provides the system of aspect 24, wherein said test sample comprises a dried blood sample. In a further aspect (26), the disclosure provides the system of aspect (25) wherein said test sample comprises dried blood in or on filter paper. In a further aspect (27), the disclosure provides the system of aspect (15), wherein said dissolving buffer, or a concentrate thereof, is provided in a first container and said lysis buffer, or a concentrate thereof, is provided in a second container. In a further aspect (28), the disclosure provides the system of any one of aspects 1-27, wherein said system comprises a reaction mixture. In a further aspect (29), the disclosure provides a method of purifying nucleic acid from a sample, comprising: contacting a sample with a dissolving and/or lysis buffer of the system of any of claims 1-28.

In a further aspect (30), the disclosure provides a method of purifying nucleic acid from a sample, comprising: contacting a sample with a dissolving buffer of any of aspects 1-12 or 15-28 to generate a dissolved sample; and contacting said dissolved sample with said lysis buffer.

In a further aspect (31), the disclosure provides the method of any one of aspects 1-22, further comprising a test sample. In an additional aspect (32), the disclosure provides the method of aspect 29 or 30 wherein said sample is a blood sample, a dried blood sample, or a whole blood sample. In an additional aspect (33) the disclosure provides the method of aspect 29 or 30 further comprising the step of treating said sample with said nucleic acid capture solid support or magnetic bead or paramagnetic bead to generate support-bound nucleic acid. In an additional aspect (34) the disclosure provides the method of aspect (33) further comprising the step of washing said support-bound nucleic acid with a wash solution. In an aspect (34) the wash solution may comprise ethanol and the ethanol may be 70% by volume ethanol. In an aspect (35), the disclosure provides a method of aspect (31) further comprising the step of contacting said support-bound nucleic acid with an elution buffer to generate eluted nucleic acid. In an aspect (36) the elution buffer of aspect (35) may comprise TE buffer.

In a further aspect (36), the disclosure provides a method according to any one of the previous aspects further comprising the step of analyzing said eluted nucleic acid. In a further aspect (37) the disclosure provides a method according to any one of the previous aspects wherein said nucleic acid comprises DNA, which may be genomic DNA.

### EXAMPLES

The compositions and methods described herein find use with a wide variety of samples. In some embodiments, the sample is from an animal. In some embodiments, the animal is a human (e.g., a human, adult or human neonate). In some embodiments, the animal is a non-human (e.g., a companion animal, livestock, rodent, infectious disease vector carrier, etc.). In some embodiments, the sample is a biological sample. In some embodiments, the sample is an environmental sample (e.g., water, soil, etc.). In some embodiments, the biological sample is a tissue or fluid sample. In some embodiments, the fluid sample comprises blood, a blood component, or a blood product (e.g., plasma, serum, etc.). In some embodiments, the blood sample is a dried blood sample. In some embodiments, the dried blood sample is a dried blood spot (DBS). In some embodiments, the dried blood spot comprises a blood sample blotted and dried on filter paper or any other substrate (e.g., a Guthrie card). In some embodiments, control samples (e.g., positive or negative controls) are provided or process along with a test sample.

Dried blood spots can be collected via any suitable technique. In some embodiments, dried blood spots are collected by applying one to several drops of blood, drawn from a subject (e.g., via lancet from the finger, heel, or toe) on filter paper. The blood is allowed to thoroughly saturate the paper and is dried (e.g., air dried). Specimens may be stored in low gas-permeability plastic bags with desiccant and kept at ambient temperature until ready for further processing. When ready for processing a technician separates a small portion (e.g., disc) of paper containing the sample (e.g., using an automated or manual hole punch). The small sample-containing portion may be added to a vessel (e.g., tube, vial, dish, etc.) and processed by the methods described herein. Alternatively, a buffer (e.g., dissolving buffer) may be flushed through the filter paper to remove desired sample. Materials and standards for dried blood spot collection are described in Hannon et al., (1997) Blood Collection on Filter Paper for Neonatal Screening Programs, 3rd edition, approved standard, National Committee for Clinical Laboratory Standards Document A4A3. National Committee for Clinical Laboratory Standards, Wayne, Pa., herein incorporated by reference in its entirety.

### EXAMPLES

### Example 1

This example provides protocols employing embodiments of the technology described herein that is suitable for use with different types of samples. These particular examples employ dried blood spot samples (example 1A) and whole blood samples (example 1B).

### Example 1A) Dried Blood Spot Samples

1) Place 3 punches of dried blood spots with 3 mm diameter in a 1.5 ml microcentrifuge tube and add 75 μl of dissolving buffer (10 mM Tris, 0.1 mM EDTA.4Na, 3% N-Lauroylsarcosine sodium salt, pH 8.0) and 1.5 μl of proteinase K. Vigorously mix by vortexing at maximum speed.

2) Incubate for 30 minutes at 56° C. Vigorously mix for 3 seconds by vortexing at a maximum speed every 10 minutes.

3) Briefly centrifuge to spin down the solution. Collect solution into a new 1.5 ml microcentrifuge tube.

4) Add 85 μl lysis buffer (5M guanidine thiocyanate, and 104 mM Na₂HPO₄.12H₂O, pH8.7) and mix by vortexing.

5) Add 95 μl anhydrous ethanol and mix by vortexing.

6) Vortex the beads at maximum speed until homogenous (the bead solution precipitates easily, so ensure they are fully mixed before usage). While swirling, add 5 μl of beads into the solution. Pipette up and down at least 20 times until fully mixed.

7) Vigorously mix for 3 seconds by vortexing at maximum speed and incubate for 5 minutes. Invert the tube 5 times every minute.

8) Briefly centrifuge to spin down the solution. Use a magnetic stand to separate the beads from the liquid until the liquid is totally clear. Carefully remove the liquid (avoid disrupting the beads as much as possible).

9) Prepare DBS Washing Buffer as below for each reaction. Scale up the volume if needed (prepare fresh DBS Washing Buffer before each usage):

DBS Wash Buffer: 37.5 μl dissolving buffer; 42.5 μl lysis buffer; 47.5 μl anhydrous ethanol.

10) Remove the tube from the magnetic stand and add 100 μl DBS Washing Buffer.

Vigorously mix for 3 seconds by vortexing until fully mixed.

11) Use a magnetic stand (a stand having a magnet 18) to separate the beads from the liquid until the liquid is totally clear. Carefully remove the clear liquid.

12) Remove the tube from the magnetic stand and add 100 μl 70% ethanol washing buffer. Vigorously mix for 3 seconds by vortexing until fully mixed.

13) Use the magnetic stand to separate the beads from the liquid until the liquid is totally clear. Carefully remove the clear liquid.

14) Repeat steps 12 and 13 twice for a total of three washes. Briefly centrifuge to spin down the solution. Use the magnetic stand to separate the beads from the liquid until the liquid is totally clear. Carefully remove the clear liquid (as much as possible).

15) Air dry for 5-10 minutes at room temperature (do not dry for too long, as this may decrease yields).

16) Remove the tube from the magnetic stand and add 50-150 μl pre-heated elution buffer (TE buffer). Pipette up and down until fully mixed.

17) Incubate at 65° C. for 5 minutes.

18) Briefly centrifuge to spin down the solution. Use the magnetic stand to separate beads from the liquid until the liquid is totally clear. Save the liquid in a new microcentrifuge tube for subsequent use/analysis or store at −20° C. for later use.

### Example 1B) Whole Blood Samples

1) Mix 1-25 μl fresh whole blood with 85 μl lysis buffer (5M guanidine thiocyanate, and 104 mM Na₂HPO₄.12H₂O, pH 8.7) by vortexing. Incubate at room temperature for 5 minutes.

2) Add 95 μl anhydrous ethanol and immediately mix by vortexing.

3) Vortex the beads at maximum speed until homogenous (the bead solution precipitates easily, so ensure they are fully mixed before usage). While swirling, add 10 μl of beads into the solution. Pipette up and down at least 20 times until fully mixed.

4) Vigorously mix for 3 seconds by vortexing at maximum speed and incubate for 5 minutes. Invert the tube 5 times every minute.

5) Briefly centrifuge to spin down the solution. Use a magnetic stand to separate the beads from the liquid until the liquid is totally clear. Carefully remove the liquid (avoid disrupting the beads as much as possible).

6) Prepare Whole Blood Washing Buffer as below for each reaction. Scale up the volume if needed (prepare fresh Whole Blood Washing Buffer before each usage; reprepare if crystals have formed):

51 μl lysis buffer; 57 μl anhydrous ethanol.

7) Remove the tube from the magnetic stand and add 100 μl Whole Blood Washing Buffer. Vigorously mix for 3 seconds by vortexing until fully mixed.

8) Use the magnetic stand to separate the beads from the liquid until the liquid is totally clear. Carefully remove the clear liquid.

9) Remove the tube from the magnetic stand and add 100 μl 70% ethanol washing buffer. Vigorously mix for 3 seconds by vortexing until fully mixed.

10) Use the magnetic stand to separate the beads from the liquid until the liquid is totally clear. Carefully remove the clear liquid.

11) Repeat steps 9 and 10 twice for a total of three washes. Briefly centrifuge to spin down the solution. Use the magnetic stand to separate the beads from the liquid until the liquid is totally clear. Carefully remove the clear liquid (as much as possible).

12) Air dry for 5-10 minutes at room temperature (do not dry for too long, as this may decrease yields).

13) Remove the tube from the magnetic stand and add 50-150 μl pre-heated elution buffer (TE buffer). Pipette up and down until fully mixed.

14) Incubate at 65° C. for 5 minutes.

15) Briefly centrifuge to spin down the solution. Use the magnetic stand to separate beads from the liquid until the liquid is totally clear. Save the liquid in a new microcentrifuge tube for subsequent use/analysis or store at −20° C. for later use.

### Example 2—Analysis of DBS

This example compares embodiments of the technology described herein in side-by-side experiments against leading commercial products using dried blood spot samples. The technology described herein provide unexpected and surprising yields of high quality nucleic acid.

gDNA was purified using a protocol described in Example 1A. Benchmark comparison using QIAamp DNA mini kit from Qiagen and DNA IQ system from Promega were conducted in parallel. Three punched pieces of DBS with 3 mm diameter were incubated in 76.5 μl dissolving buffer/proteinase K at 56° C. for 30 minutes (10 mM Tris, 0.1 mM EDTA.4Na, 3% N-Lauroylsarcosine sodium salt, pH 8.0). Samples were vortexed vigorously every 10 minutes to obtaining higher gDNA yield. The dissolving solution was combined with 85 μl lysis buffer (5M guanidine thiocyanate, and 104 mM Na₂HPO₄.12H₂O, pH 8.7) and 95 μl anhydrous ethanol to attain the binding environment. Next, 5 μl Q beads (MagQu Co. Ltd.) was mixed with the samples for DNA binding for 5 minutes. Beads were washed with 100 μl DBS Washing Buffer once and with 100 μl 70% ethanol three times. DNA was then eluted by 50 μl TE buffer at 65° C. for 5 minutes.

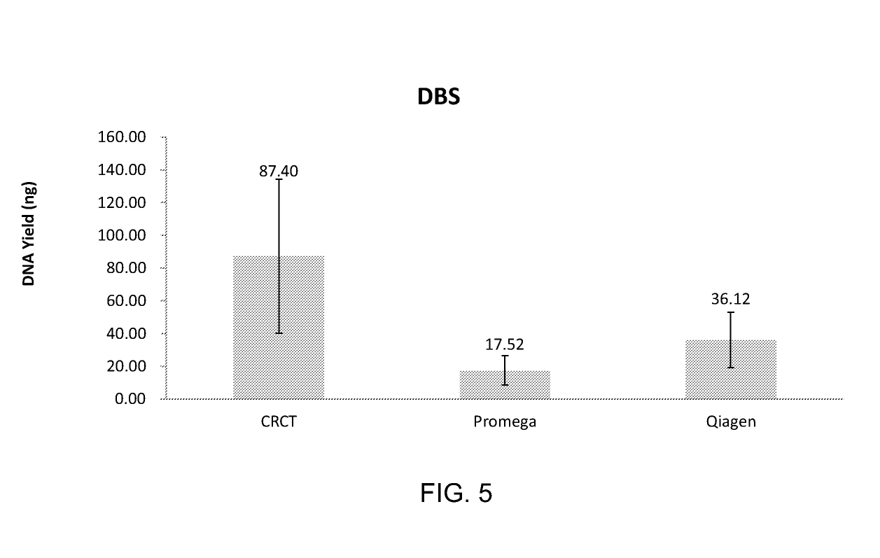

The concentration/yield of DNA was determined by QUBIT® dsDNA HS (High Sensitivity) Assay Kit and QUBIT® 2.0 Fluorometer and the quality was verified by PCR and quantitative PCR (qPCR). The results showed that the technology provided herein had more than twice the gDNA yield of QIAamp DNA Mini Kit and almost five times of that of DNA IQ System (FIG. 5). The large error bar was due to the donor to donor difference. The fold increase was quite consistent. Purified gDNA was resolved by 1% agarose gel electrophoresis. The data showed that the technology provided herein provided a clearer and more intact gDNA band than Promega and Qiagen kits.

GAPDH sequence was also amplified from 1 μl of purified gDNA (1/50 eluent) using PCR and resolved by 1% agarose gel electrophoresis. The image showed that GAPDH was amplified in all kits. To further confirm the gDNA quality, 1 μl of purified gDNA (1/50 eluent) was analyzed by real-time PCR, a more sensitive method for amplification than PCR. The result showed that the technology provided herein had the best performance as compared to the other two kits.

### Example 3—Analysis of Whole Blood Samples

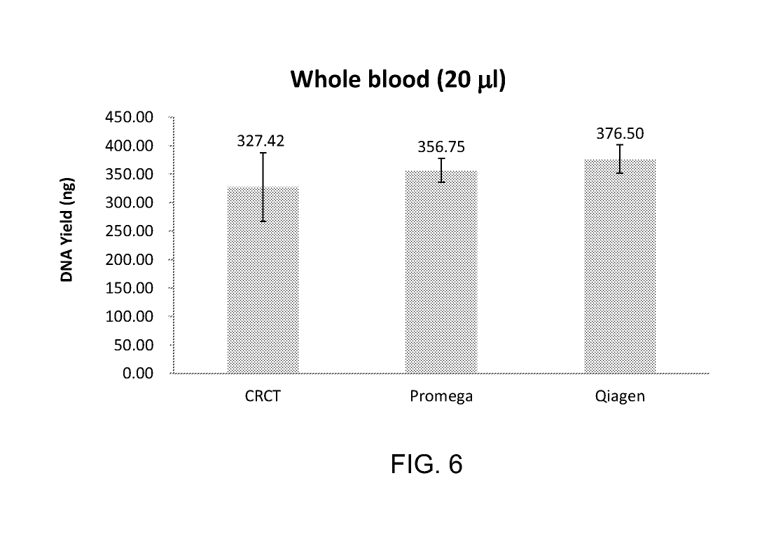

This example compares embodiments of the technology described herein in side-by-side experiments against leading commercial products using whole blood samples. The protocol described in Example 1B was employed. Parallel comparisons to the two benchmark kits were conducted as well. The concentration/yield of DNA was determined by QUBIT® dsDNA HS (High Sensitivity) Assay Kit and QUBIT® 2.0 Fluorometer, and the quality was verified by PCR and quantitative PCR (qPCR). The result showed that the technology described herein had comparable gDNA yield with QIAamp DNA Mini Kit and DNA IQ System (FIG. 6). Purified gDNA was resolved by 1% agarose gel electrophoresis. The results showed that gDNA was purified even from as low as 1 μl whole blood sample using the technology described herein. The gDNA band showed comparable intensity with Promega kit and slightly higher than Qiagen kit. GAPDH sequence was also amplified from 1 μl of purified gDNA (1/50 eluent) using PCR and resolved by 1% agarose gel electrophoresis. The image showed that GAPDH was amplified in all kits. To further confirm the gDNA quality, 1 μl of purified gDNA (1/50 eluent) from 20 μl whole blood samples was analyzed by real-time PCR. The result showed that the technology had comparable performance with the other two kits.

### Example 4—Buffer Performance

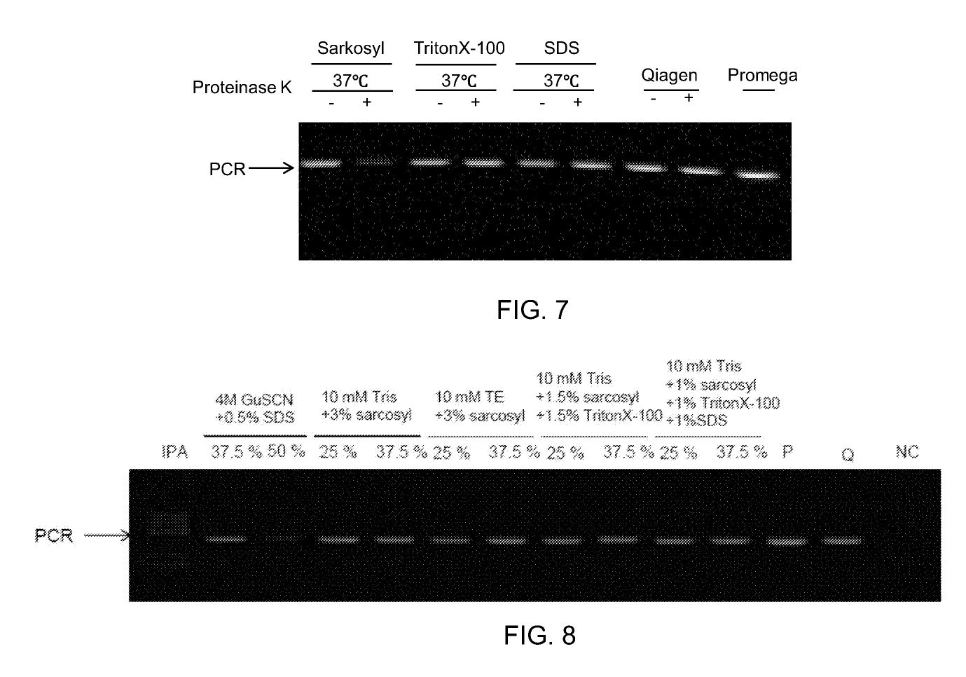

This Example describes alternative buffer formulations and their performances. In a first experiment, dissolving buffer was formulated with 10 mM Tris and alternatively 3% sarkosyl, 1% TRITON X-100, or 0.5% SDS and compared against benchmark Qiagen and Promega products. A lysis/binding buffer comprising 2.5 M guanidine thiocyanate (GuSCN), 25% isopropyl alcohol (IPA), and alternatively 0.75% sarcosyl, 0.25% TRITONX-100, or 0.125% SDS was employed. The dissolving buffer was tested with and without proteinase K. Purified nucleic acid from dried blood spots was amplified by PCR and product was run on a gel. Results are shown in FIG. 7, with each sample producing a band.

A second experiment tested alternative dissolving buffer surfactants and bases with isopropyl alcohol. Each of the following thirteen reactions were conducted (showing the mixture of dissolving buffer, lysis buffer, and binding buffer alcohol):
(1) 2.5M GuSCN+0.31% SDS+37.5% IPA;
(2) 2.5M GuSCN+0.25% SDS+50% IPA;
(3) 2.5 mM Tris+2.5M GuSCN+0.75% Sarkosyl+25% IPA;
(4) 1.25 mM Tris+2.5M GuSCN+0.375% Sarkosyl+37.5% IPA;
(5) 2.5 mM Tris+0.025mMEDTA.4Na+2.5M GuSCN+0.75% Sarkosyl+25% IPA;
(6) 1.25 mM Tris+0.0125mMEDTA.4Na+2.5M GuSCN+0.375% Sarkosyl+37.5% IPA;
(7) 2.5 mM Tris+2.5M GuSCN+0.375% Sarkosyl+0.375% TritonX-100+25% IPA;
(8) 1.25 mM Tris+2.5M GuSCN+0.1875% Sarkosyl+37.5% IPA;
(9) 2.5 mM Tris+2.5M GuSCN+0.25% Sarkosyl+0.25% TritonX-100+0.25% SDS+25% IPA;
(10) 1.25 mM Tris+2.5M GuSCN+0.125% Sarkosyl+0.125% TritonX-100+0.125% SDS+37.5% IPA;
(11) Promega kit (P);
(12) QIAGEN kit (Q); and
(13) PCR no-template negative control.

Purified nucleic acid from dried blood spots was amplified by PCR and product was run on a gel. Results are shown in FIG. 8.

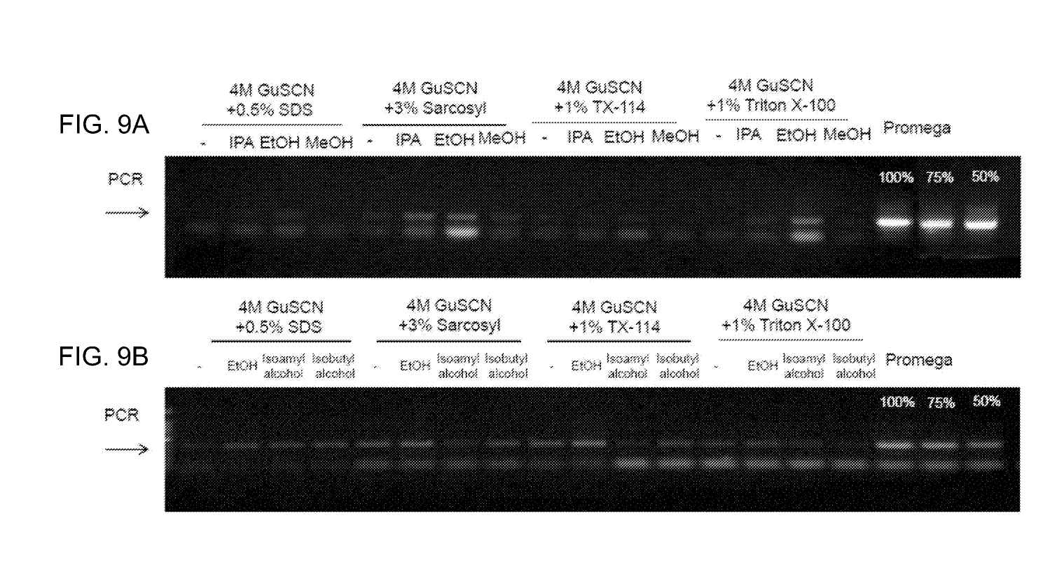

A third experiment compared alcohols used in the binding buffer along with different surfactants in the dissolving buffer. 4 M GuSCN was used in the lysis buffer. The dissolving buffer alternatively used with 0.5% SDS, 3% sarcosyl, 1% TX-114, or 1% TRITONX-100. Each was tested with IPA, ethanol, methanol, isoamyl alcohol, or isobutyl alcohol. Purified nucleic acid from dried blood spots was amplified by PCR and product was run on a gel. Results are shown in FIGS. 9A and 9B. The GuSCN, sarcosyl, ethanol combination produced the strongest band.

A fourth experiment compared different surfactants with TE-buffer in the dissolving buffer (0.5% SDS, 3% sarcosyl, 1% TX-114, 1% TRITONX-100) as compared to the Promega kit. Purified nucleic acid from dried blood spots was analyzed with the 3% sarcosyl providing the highest observed yield and each of the samples higher than the commercial product.

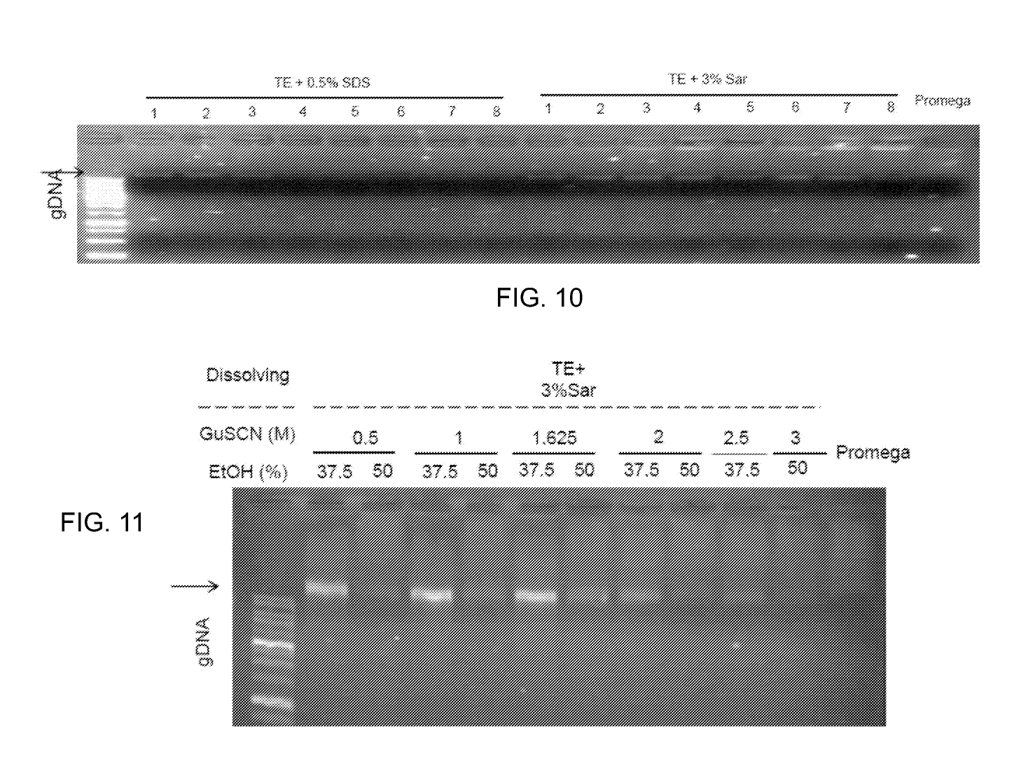

A fifth experiment compared different relative levels of dissolving buffer, lysis buffer, and alcohol in the binding buffer with either SDS or sarcosyl surfactants and different guanidine compounds. Results are shown in FIG. 10 (volumes, in order, are: dissolving buffer, lysis buffer, alcohol) from dried blood spot samples.

A sixth experiment employed 3% sarcosyl with TE buffer as the dissolving buffer and varied the concentration of GuSCN in the lysis buffer as well as varying ethanol concentration in the binding buffer. Results are shown in FIG. 11 from dried blood spot samples. QUBIT quantitation of nucleic acid showed that 1.625 M GuSCN with 37.5% ethanol provided the highest yield.

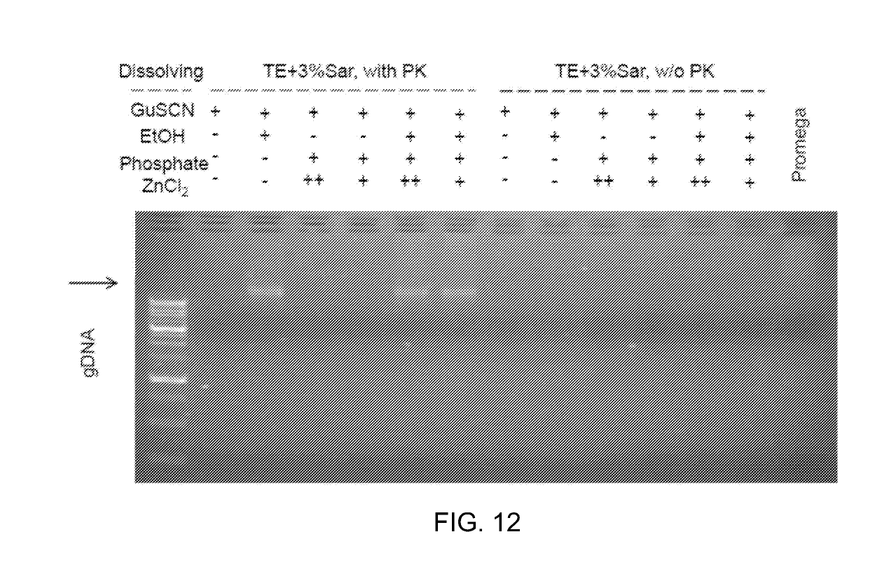

A seventh experiment employed 3% sarcosyl with TE buffer as the dissolving buffer, with and without proteinase K; GuSCN in the lysis buffer with and without salts (phosphate and ZnCl₂); and +/−ethanol in the binding buffer. Results are shown in FIG. 12 from dried blood spot samples. QUBIT quantitation of nucleic acid showed strong results for dissolving buffer with proteinase K, lysis buffer with or without salt, and ethanol.

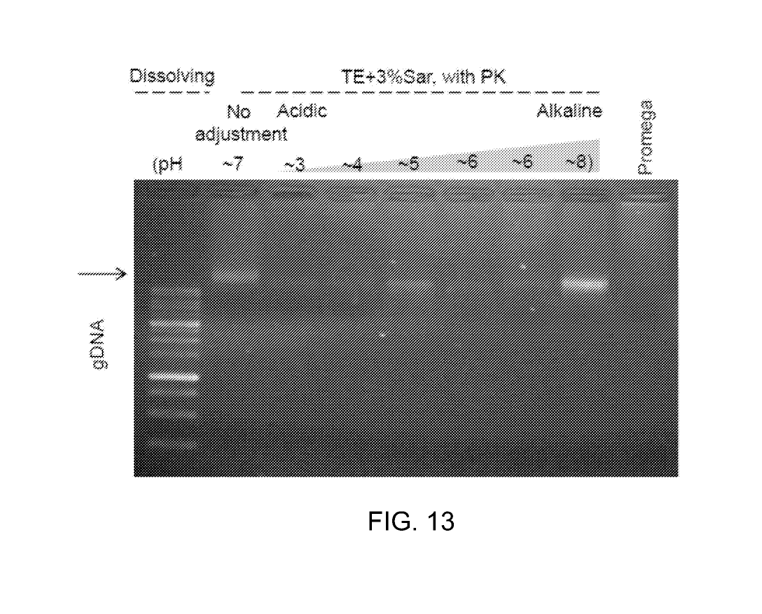

An eighth experiment employed 3% sarcosyl with TE buffer as the dissolving buffer, with proteinase K, with varying pH conditions for the lysis/binding step varying from a pH of 3 to 8. Results are shown in FIG. 13 from dried blood spot samples. A pH of 8 produced the strongest yield as shown by QUBIT quantitation of nucleic acid.

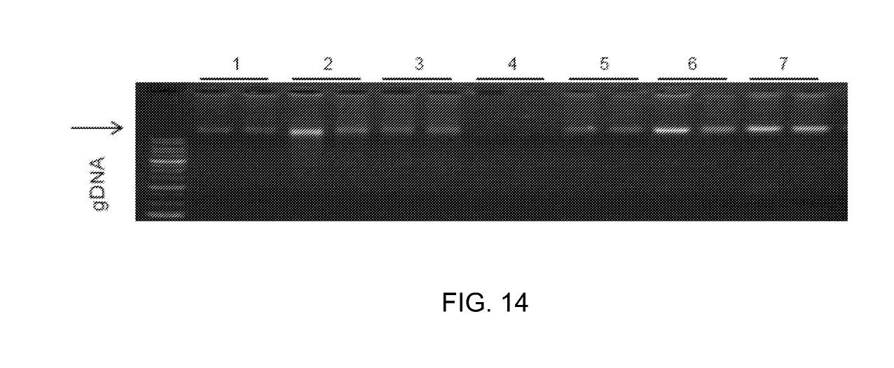

A ninth experiment tested variations in buffer compositions (sarkosyl concentration; proteinase K versus 2-mercaptoethanol; mixing methods; washing methods (W1A buffer; W2=28.5 mM sodium acetate, 66 mM ammonium acetate, 70% ethanol wash; DBSW=wash with buffer/alcohol combo); elution buffer (water versus TE buffer). Results are shown in FIG. 14 from dried blood spots. QUBIT quantitation of nucleic acid showed the best yield with the combination of: dissolving buffer (TE-buffer, 3% sarkosyl, proteinase K) and TE-elution buffer.

An additional experiment compared different paramagnetic beads. Q beads were compared to Magen beads (MagPure Forensic DAN Kitts II from Magentech). Both performed well. However, Q beads provided a higher yield of purified nucleic acid from dried blood spot samples.

1. A system comprising:
a) a dissolving buffer comprising a surfactant;
b) a lysis buffer comprising a protein denaturant; and
c) a nucleic acid capture solid support.

2. The system of claim 1, wherein said dissolving buffer further comprises a chelating agent and a base.

3. The system of claim 1, wherein said lysis buffer further comprises a salt.

4. The system of claim 1, wherein said nucleic acid capture solid support comprises a magnetic or a paramagnetic bead.

5. The system of claim 1, wherein said surfactant comprises an ionic surfactant.

6. The system of claim 5, wherein said ionic surfactant comprises an ionic surfactant derived from sarcosine.

7. The system of claim 6, wherein said surfactant comprises N-lauroylsarcosine.

8. The system of claim 7, wherein said surfactant comprises sodium [dodecanoyl(methyl)amino]acetate.

9. The system of claim 1, wherein said surfactant is present in the dissolving buffer at from 0.05-10% by volume.

10. The system of claim 9, wherein said surfactant is present in the dissolving buffer at 3% by volume.

11. The system of claim 1, wherein said protein denaturant comprises proteinase K.

12. A system comprising:
a) a lysis buffer, said lysis buffer comprising guanidine thiocyanate, guanidine hydrochloride, or combinations thereof and disodium hydrogen phosphate; and
b) a nucleic acid capture solid support.

13. The system of claim 12, further comprising a dissolving buffer comprising a surfactant, a chelating agent, and a base.

14. A system comprising:
a) a dissolving buffer comprising N-lauroylsarcosine sodium salt; and
b) a lysis buffer comprising guanidine thiocyanate.

15. The system of claim 14, wherein said dissolving buffer further comprises a base and a chelating agent.

16. The system of claim 14, wherein said dissolving buffer further comprises one or more components that denature or destroy proteins.

17. The system of claim 14, wherein said lysis buffer further comprises a salt.

18. The system of claim 14, further comprising a nucleic acid capture solid support.

19. The system of claim 18, wherein said a nucleic acid capture solid support comprises a magnetic bead or a paramagnetic bead.

20. The system of claim 1, further comprising one or more wash buffers.

21. The system of claim 1, further comprising a magnet.

22. The system of claim 21, wherein said test sample comprises a blood sample.

23. The system of claim 22, wherein said test sample comprises a dried blood sample.

24. The system of claim 22, wherein said test sample comprises dried blood in filter paper.

25. A method of purifying nucleic acid from a sample, the method comprising:
contacting a sample with a dissolving buffer of claim 1 to generate a dissolved sample; and
contacting said dissolved sample with said lysis buffer.
